(19) United States
(12) Patent Application Publication
(10) Pub. No.: US 2024/0147928 A1
(43) Pub. Date: May 9, 2024

(54) CAMPHOR-FREE AND HIGH HERNANDULCIN PHYLA DULCIS PLANTS AND METHODS THEREOF

(71) Applicant: THE TEXAS A&M UNIVERSITY SYSTEM, College Station, TX (US)

(72) Inventors: Godson O. OSUJI, College Station, TX (US); Laura E. CARSON, College Station, TX (US); Ming GAO, College Station, TX (US); Aruna WEERASOORIYA, College Station, TX (US); Peter A. AMPIM, College Station, TX (US); Sela WOLDENSENBET, College Station, TX (US); Paul JOHNSON, College Station, TX (US)

(21) Appl. No.: 18/548,398

(22) PCT Filed: Mar. 2, 2022
(86) PCT No.: PCT/US2022/018430

§ 371 Date: Aug. 30, 2023

Related U.S. Application Data
(60) Provisional application No. 63/155,375, filed on Mar. 2, 2021.

(51) Int. Cl. A01H 3/04; A01N 59/08; A01N 59/26

(52) U.S. Cl. CPC A01H 3/04; A01N 59/08; A01N 59/26

(57) ABSTRACT

The present disclosure provides methods for producing Phyla dulcis plants and plant parts that are substantially camphor-free and also have an increased level of hernandulcin. Phyla dulcis plants and plant parts, as well as compositions comprising the same, are also provided herein. The present disclosure provides methods that utilize the regulation of the terpenoid biochemical pathways of P. dulcis as opposed to previous methods that depend on chemical synthesis or genetic, enzyme, microbial, and plant tissue engineering.

### CROSS-REFERENCE TO RELATED APPLICATIONS

This application claims the benefit under 35 USC § 119(e) of U.S. Provisional Application Ser. No. 63/155,375, filed on Mar. 2, 2021, the entire disclosure of which is incorporated herein by reference.

### BACKGROUND AND SUMMARY OF THE INVENTION

With each passing year, consumers become more knowledgeable to the principles of healthy living and the practices of healthy eating. In this regard, there has been a substantial increase in the demand for naturally extracted zero-calorie sweeteners as consumers are continually steered away from sugar to low-calorie alternatives. The expanding consumption of non-sugar sweeteners in the United States has been estimated to account for about $1.5 billion.

Plant-derived natural sweeteners offer a solution to this interest. Given its structural simplicity and high sweetness potency as being 1000 times sweeter than sucrose, hernandulcin has been a focus of research. Hernandulcin is a chemical compound derived from Central American plant Phyla dulcis (Trey.) Mold (Verbenaceae). However, despite considerable efforts to maximize the efficiency of extraction from the Phyla dulcis plant, yields of hernandulcin have remained very low and also contaminated with camphor that gives an undesirable taste profile. Attempts to increase hernandulcin and eliminate camphor via metabolic engineering, hydro-distillation, supercritical fluid extraction, microbial degradation, and root tissue culture synthesis have proven unsuccessful. Further, chemical synthesis of hernandulcin resulted in production of varieties that were not sweet. Therefore, there exists a need for an effective way to produce P. dulcis plants and plant parts that have high concentrations of hernandulcin and, at the same time, low concentrations of camphor.

Accordingly, the present disclosure provides methods for producing Phyla dulcis plants and plant parts that are substantially camphor-free and also have an increased level of hernandulcin. Phyla dulcis plants and plant parts, as well as compositions comprising the same, are also provided herein.

The methods and compositions provided herein provide several benefits that have never been achieved in the field of hernandulcin. Importantly, the disclosed methods provide Phyla dulcis plants and plant parts that can achieve an increased level of hernandulcin while, at the same time, being substantially camphor-free. As opposed to methods that depend on chemical synthesis or genetic, enzyme, microbial, and plant tissue engineering, methods described herein utilize the regulation of the terpenoid biochemical pathways of P. dulcis that provide practical simplicity. Moreover, hernandulcin is naturally synthesized by plants as opposed to chemically synthesized sweeteners such as saccharin, cyclamate, neohesperidin, dihydrochalcone, asparmate, acesulfame K, sucralose, neotame, and advantame.

Further, the methods are economically sustainable because they utilize smaller concentrations of inorganic salts instead of larger weights of commercial mineral fertilizers. As such, the described methods are agriculturally friendly to limited resource farmers and can be cultivated on healthy soil. The described methods are also amenable to year-round greenhouse scale-up commercial production, which can be utilized to meet the 425,000 hectares competition threshold of sugar cane agriculture.

As a result, the embodiments of the present disclosure can provide hernandulcin sugar substitutes as well as new food and beverage products. Furthermore, P. dulcis extracts can potentially be useful in the dietary management of diabetes and obesity, as well as application in other health care aspects.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows Phyla dulcis proportionation of mono- and sesqui-terpene pathways. The figure demonstrates the zigzag molecular chemistry machine complex for hernandulcin.

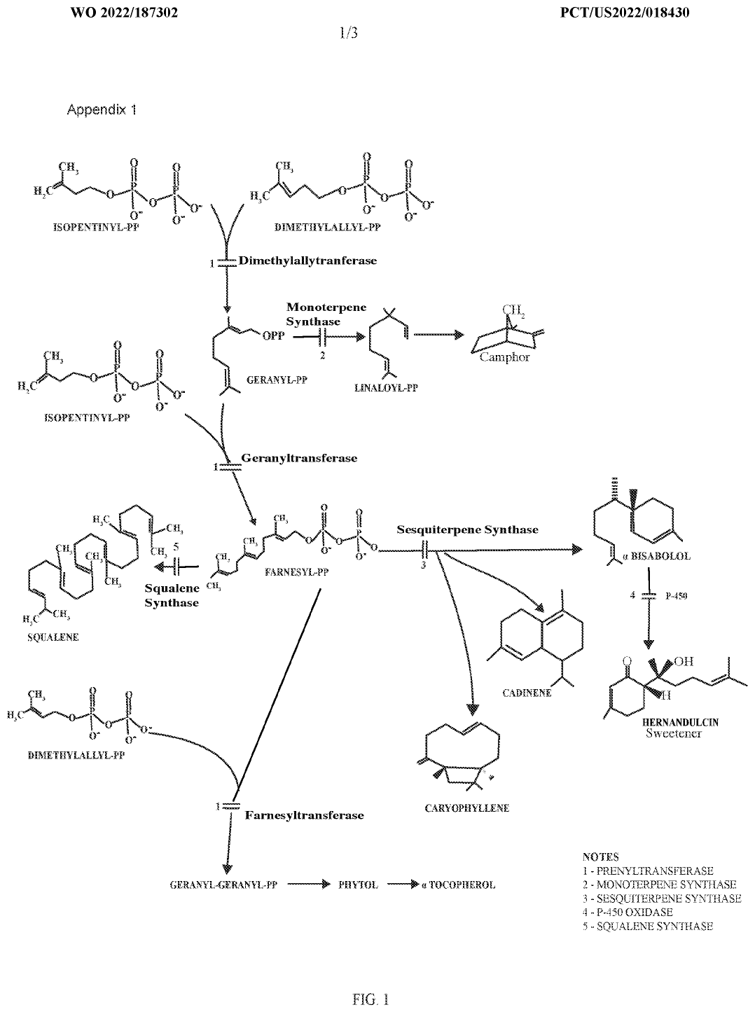

FIG. 2 shows a depiction of P. dulcis growing in a field plot.

FIG. 3 shows a depiction of raised bed cultivation of P. dulcis.

### DETAILED DESCRIPTION

Various embodiments of the invention are described herein as follows. In an illustrative aspect, a method of producing a substantially camphor-free Phyla dulcis plant is provided. The method comprises the step of treating a Phyla dulcis plant with a mineral salt solution, wherein the treated Phyla dulcis plant grows to form the substantially camphor-free Phyla dulcis plant. As used herein, the term “substantially camphor-free” refers to a plant, plant part, or any other composition with a low presence of camphor. For instance, “substantially camphor-free” can mean the presence of camphor at a level of less than 0.001% (w/w), or less than 0.002% (w/w), or less than 0.003% (w/w), or less than 0.004% (w/w), or less than 0.005% (w/w), or less than 0.006% (w/w), or less than 0.007% (w/w), or less than 0.008% (w/w), or less than 0.009% (w/w), or less than 0.01% (w/w).

In an embodiment, the substantially camphor-free Phyla dulcis plant comprises an increased level of hernandulcin. Hernandulcin is known in the art as a sweetener which is 1000 times sweeter than sucrose. Hernandulcin has a molecular formula of C₁₅H₂₄O₂ and its chemical structure is:

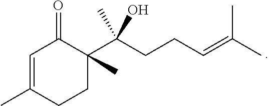 [SMILES: CC(C)=CCC[C@](C)(O)[C@]1(C)CCC(C)=CC1=O]

In an embodiment, the increased level of hernandulcin is in comparison to a comparative Phyla dulcis plant comprising camphor. In an embodiment, the increased level of hernandulcin is in comparison to a comparative Phyla dulcis plant not treated with a mineral salt solution. In an embodiment, the increased level of hernandulcin is greater than 7% w/w. In an embodiment, the increased level of hernandulcin is greater than 6.5% w/w. In an embodiment, the increased level of hernandulcin is greater than 6% w/w. In an embodiment, the increased level of hernandulcin is greater than 5.5% w/w. In an embodiment, the increased level of hernandulcin is greater than 5.3% w/w. In an embodiment, the increased level of hernandulcin is greater than 5% w/w. In an embodiment, the increased level of hernandulcin is greater than 4.5% w/w. In an embodiment, the increased level of hernandulcin is greater than 4% w/w.

In an embodiment, the substantially camphor-free Phyla dulcis plant comprises an increased biomass in comparison to a comparative Phyla dulcis plant comprising camphor. In an embodiment, the substantially camphor-free Phyla dulcis plant comprises an increased biomass in comparison to a comparative Phyla dulcis plant not treated with a mineral salt solution.

In an embodiment, the substantially camphor-free Phyla dulcis plant comprises an antioxidant. In an embodiment, the antioxidant is a shikimal-based antioxidant.

In an embodiment, the mineral salt solution comprises an element selected from the group consisting of K, Cl, Na, P, O, N, H, S, and any combination thereof. In an embodiment, the mineral salt solution comprises an element selected from the group consisting of K, P, N, S, and any combination thereof. In an embodiment, the mineral salt solution comprises a formulation selected from the group consisting of KCl, Na₃PO₄, NH₄Cl, Na₂SO₄, and any combination thereof.

In an embodiment, the mineral salt solution comprises KCl. In an embodiment, the KCl is present at 4 mM. In an embodiment, the KCl is present at 8 mM.

In an embodiment, the mineral salt solution comprises Na₃PO₄. In an embodiment, the Na₃PO₄ is present at 20 mM. In an embodiment, the Na₃PO₄ is present at 40 mM.

In an embodiment, the mineral salt solution comprises NH₄Cl. In an embodiment, the NH₄Cl is present at 25 mM.

In an embodiment, the mineral salt solution comprises Na₂SO₄. In an embodiment, the Na₂SO₄ is present at 50 mM.

In an embodiment, the mineral salt solution comprises KKPP, wherein KKPP comprises KCl and Na₃PO₄. In an embodiment, the KCl is provided at 8 mM and the Na₃PO₄ is provided at 40 mM. In an embodiment, the mineral salt solution has a stoichiometric ratio of 8 mM KCl:40 mM Na₃PO₄.

In an embodiment, the mineral salt solution comprises NPK, wherein NPK comprises NH₄Cl, Na₃PO₄, and KCl. In an embodiment, the NH₄Cl is provided at 25 mM, the Na₃PO₄ is provided at 20 mM, and the KCl is provided at 4 mM. In an embodiment, the mineral salt solution has a stoichiometric ratio of 25 mM NH₄Cl:20 mM Na₃PO₄:4 mM KCl.

In an embodiment, the mineral salt solution comprises KS, wherein KS comprises KCl and Na₂SO₄. In an embodiment, the KCl is provided at 4 mM and the Na₂SO₄ is provided at 50 mM. In an embodiment, the mineral salt solution has a stoichiometric ratio of 4 mM KCL:50 mM Na₂SO₄.

In an embodiment, the treated Phyla dulcis plant grows on soil. In an embodiment, the soil is healthy soil. In an embodiment, the soil is healthy soil comprising a combination of i) growing mix and ii) topsoil comprising organic matter. In an embodiment, the growing mix and the topsoil are present at a ratio of 3:2 (v/v).

In an embodiment, the Phyla dulcis plant is present in a greenhouse. In an embodiment, the Phyla dulcis plant is present in a bed. In an embodiment, the Phyla dulcis plant is present in a field plot bed. In an embodiment, the Phyla dulcis plant is present in a raised bed. In an embodiment, the Phyla dulcis plant is present in a garden. In an embodiment, the Phyla dulcis plant is present in a raised bed in a garden.

In an illustrative aspect, a substantially camphor-free Phyla dulcis plant is provided, in which the plant is produced according to the described methods. In an embodiment, the substantially camphor-free Phyla dulcis plant comprises an increased level of hernandulcin.

In an illustrative aspect, a substantially camphor-free Phyla dulcis plant part is provided, in which the plant part is produced according to the described methods. In an embodiment, the substantially camphor-free Phyla dulcis plant part comprises an increased level of hernandulcin. In an embodiment, the plant part is selected from the group consisting of a seed, a flower, a leaf, and a stem. In an embodiment, the plant part is a seed. In an embodiment, the plant part is a flower. In an embodiment, the plant part is a leaf. In an embodiment, the plant part is a stem.

In an illustrative aspect, a method of producing a final Phyla dulcis plant or plant part comprising an increased level of hernandulcin is provided. The method comprises the step of treating a Phyla dulcis plant or plant part with a mineral salt solution, wherein the treated Phyla dulcis plant or plant part grows to form the final Phyla dulcis plant or plant part comprising an increased level of hernandulcin.

In an embodiment, the final Phyla dulcis plant or plant part is substantially free of camphor. In an embodiment, the final Phyla dulcis plant or plant part comprises an increased level of hernandulcin. In an embodiment, the increased level of hernandulcin is in comparison to a comparative Phyla dulcis plant or plant part comprising camphor. In an embodiment, the increased level of hernandulcin is in comparison to a comparative Phyla dulcis plant or plant part not treated with a mineral salt solution.

In an embodiment, the increased level of hernandulcin is greater than 7% w/w. In an embodiment, the increased level of hernandulcin is greater than 6.5% w/w. In an embodiment, the increased level of hernandulcin is greater than 6% w/w. In an embodiment, the increased level of hernandulcin is greater than 5.5% w/w. In an embodiment, the increased level of hernandulcin is greater than 5.3% w/w. In an embodiment, the increased level of hernandulcin is greater than 5% w/w. In an embodiment, the increased level of hernandulcin is greater than 4.5% w/w. In an embodiment, the increased level of hernandulcin is greater than 4% w/w.

In an embodiment, the substantially camphor-free Phyla dulcis plant comprises an increased biomass in comparison to a comparative Phyla dulcis plant comprising camphor. In an embodiment, the substantially camphor-free Phyla dulcis plant comprises an increased biomass in comparison to a comparative Phyla dulcis plant not treated with a mineral salt solution.

In an embodiment, the final Phyla dulcis plant or plant part comprises an antioxidant. In an embodiment, the antioxidant is a shikimal-based antioxidant.

In an embodiment, the mineral salt solution comprises an element selected from the group consisting of K, Cl, Na, P, O, N, H, S, and any combination thereof. In an embodiment, the mineral salt solution comprises an element selected from the group consisting of K, P, N, S, and any combination thereof. In an embodiment, the mineral salt solution comprises a formulation selected from the group consisting of KCl, Na₃PO₄, NH₄Cl, Na₂SO₄, and any combination thereof.

In an embodiment, the mineral salt solution comprises KCl. In an embodiment, the KCl is present at 4 mM. In an embodiment, the KCl is present at 8 mM.

In an embodiment, the mineral salt solution comprises Na₃PO₄. In an embodiment, the Na₃PO₄ is present at 20 mM. In an embodiment, the Na₃PO₄ is present at 40 mM.

In an embodiment, the mineral salt solution comprises NH₄Cl. In an embodiment, the NH₄Cl is present at 25 mM. In an embodiment, the mineral salt solution comprises Na₂SO₄. In an embodiment, the Na₂SO₄ is present at 50 mM.

In an embodiment, the mineral salt solution comprises KKPP, wherein KKPP comprises KCl and Na₃PO₄. In an embodiment, the KCl is provided at 8 mM and the Na₃PO₄ is provided at 40 mM. In an embodiment, the mineral salt solution has a stoichiometric ratio of 8 mM KCl:40 mM Na₃PO₄.

In an embodiment, the mineral salt solution comprises NPK, wherein NPK comprises NH₄Cl, Na₃PO₄, and KCl. In an embodiment, the NH₄Cl is provided at 25 mM, the Na₃PO₄ is provided at 20 mM, and the KCl is provided at 4 mM. In an embodiment, the mineral salt solution has a stoichiometric ratio of 25 mM NH₄Cl:20 mM Na₃PO₄:4 mM KCl.

In an embodiment, the mineral salt solution comprises KS, wherein KS comprises KCl and Na₂SO₄. In an embodiment, the KCl is provided at 4 mM and the Na₂SO₄ is provided at 50 mM. In an embodiment, the mineral salt solution has a stoichiometric ratio of 4 mM KCL:50 mM Na₂SO₄.

In an embodiment, the treated Phyla dulcis plant grows on soil. In an embodiment, the soil is healthy soil. In an embodiment, the soil is healthy soil comprising a combination of i) growing mix and ii) topsoil comprising organic matter. In an embodiment, the growing mix and the topsoil are present at a ratio of 3:2 (v/v).

In an embodiment, the Phyla dulcis plant is present in a greenhouse. In an embodiment, the Phyla dulcis plant is present in a bed. In an embodiment, the Phyla dulcis plant is present in a field plot bed. In an embodiment, the Phyla dulcis plant is present in a raised bed. In an embodiment, the Phyla dulcis plant is present in a garden. In an embodiment, the Phyla dulcis plant is present in a raised bed in a garden.

In an illustrative aspect, a Phyla dulcis plant is provided, in which the plant is produced according to the described methods. In an embodiment, the Phyla dulcis plant comprises an increased level of hernandulcin.

In an illustrative aspect, a Phyla dulcis plant part is provided, in which the plant part is produced according to the described methods. In an embodiment, the Phyla dulcis plant part comprises an increased level of hernandulcin. In an embodiment, the plant part is selected from the group consisting of a seed, a flower, a leaf, and a stem. In an embodiment, the plant part is a seed. In an embodiment, the plant part is a flower. In an embodiment, the plant part is a leaf. In an embodiment, the plant part is a stem.

In an illustrative aspect, a composition comprising hernandulcin is provided, wherein the hernandulcin is produced according to the described methods.

In an embodiment, the composition is configured for inclusion in a beverage. In an embodiment, the beverage is selected from the group consisting of a soft drink beverage, a bottled beverage, an alcoholic beverage, a non-alcoholic beverage, a weight-loss beverage, and water.

In an embodiment, the composition is configured for inclusion in a confectionary. In an embodiment, the confectionary is selected from the group consisting of a jelly, a syrup, a honey, a candy, a chewing gum, and any combination thereof.

In an embodiment, the composition is configured for inclusion in a food. In an embodiment, the food is selected from the group consisting of a bread, a cake, a cookie, a salad dressing, a corn flake, a rice flake, and any combination thereof.

In an embodiment, the composition is a dietary supplement.

In an embodiment, the composition is configured for inclusion in a formulation selected from the group consisting of a sugar-free malt drink, a sugar-free soft drink, a sugar-free beer, a sugar-free ice cream, a sugar-free yogurt, a sugar-free toothpaste, a sugar-free iced tea, a sugar-free jelly, a sugar-free candy, a sugar-free bottled water, a sugar-free fruit juice, a sugar-free marmalade, a sugar-free chewing gum, a sugar-free mouth wash, and a sugar-free tomato ketchup. In an embodiment, the composition is configured for inclusion in a sugar-free malt drink. In an embodiment, the composition is configured for inclusion in a sugar-free soft drink. In an embodiment, the composition is configured for inclusion in a sugar-free beer. In an embodiment, the composition is configured for inclusion in a sugar-free ice cream. In an embodiment, the composition is configured for inclusion in a sugar-free yogurt. In an embodiment, the composition is configured for inclusion in a sugar-free toothpaste. In an embodiment, the composition is configured for inclusion in a sugar-free iced tea. In an embodiment, the composition is configured for inclusion in a sugar-free jelly. In an embodiment, the composition is configured for inclusion in a sugar-free candy. In an embodiment, the composition is configured for inclusion in a sugar-free bottled water. In an embodiment, the composition is configured for inclusion in a sugar-free fruit juice. In an embodiment, the composition is configured for inclusion in a sugar-free marmalade. In an embodiment, the composition is configured for inclusion in a sugar-free chewing gum. In an embodiment, the composition is configured for inclusion in a sugar-free mouth wash. In an embodiment, the composition is configured for inclusion in a sugar-free tomato ketchup.

The following numbered embodiments are contemplated and are non-limiting:

1. A method of producing a substantially camphor-free Phyla dulcis plant, said method comprising the step of treating a Phyla dulcis plant with a mineral salt solution, wherein the treated Phyla dulcis plant grows to form the substantially camphor-free Phyla dulcis plant.
2. The method of clause 1, any other suitable clause, or any combination of suitable clauses, wherein the substantially camphor-free Phyla dulcis plant comprises an increased level of hernandulcin.
3. The method of clause 2, any other suitable clause, or any combination of suitable clauses, wherein the increased level of hernandulcin is in comparison to a comparative Phyla dulcis plant comprising camphor.
4. The method of clause 2, any other suitable clause, or any combination of suitable clauses, wherein the increased level of hernandulcin is in comparison to a comparative Phyla dulcis plant not treated with a mineral salt solution.
5. The method of clause 2, any other suitable clause, or any combination of suitable clauses, wherein the increased level of hernandulcin is greater than 7% w/w.
6. The method of clause 2, any other suitable clause, or any combination of suitable clauses, wherein the increased level of hernandulcin is greater than 6.5% w/w.
7. The method of clause 2, any other suitable clause, or any combination of suitable clauses, wherein the increased level of hernandulcin is greater than 6% w/w.
8. The method of clause 2, any other suitable clause, or any combination of suitable clauses, wherein the increased level of hernandulcin is greater than 5.5% w/w.
9. The method of clause 2, any other suitable clause, or any combination of suitable clauses, wherein the increased level of hernandulcin is greater than 5.3% w/w.
10. The method of clause 2, any other suitable clause, or any combination of suitable clauses, wherein the increased level of hernandulcin is greater than 5% w/w.
11. The method of clause 2, any other suitable clause, or any combination of suitable clauses, wherein the increased level of hernandulcin is greater than 4.5% w/w.
12. The method of clause 2, any other suitable clause, or any combination of suitable clauses, wherein the increased level of hernandulcin is greater than 4% w/w.
13. The method of clause 1, any other suitable clause, or any combination of suitable clauses, wherein the substantially camphor-free Phyla dulcis plant comprises an increased biomass in comparison to a comparative Phyla dulcis plant comprising camphor.
14. The method of clause 1, any other suitable clause, or any combination of suitable clauses, wherein the substantially camphor-free Phyla dulcis plant comprises an increased biomass in comparison to a comparative Phyla dulcis plant not treated with a mineral salt solution.
15. The method of clause 1, any other suitable clause, or any combination of suitable clauses, wherein the substantially camphor-free Phyla dulcis plant comprises an antioxidant.
16. The method of clause 15, any other suitable clause, or any combination of suitable clauses, wherein the antioxidant is a shikimal-based antioxidant.
17. The method of clause 1, any other suitable clause, or any combination of suitable clauses, wherein the mineral salt solution comprises an element selected from the group consisting of K, Cl, Na, P, O, N, H, S, and any combination thereof.
18. The method of clause 1, any other suitable clause, or any combination of suitable clauses, wherein the mineral salt solution comprises an element selected from the group consisting of K, P, N, S, and any combination thereof.
19. The method of clause 1, any other suitable clause, or any combination of suitable clauses, wherein the mineral salt solution comprises a formulation selected from the group consisting of KCl, Na₃PO₄, NH₄Cl, Na₂SO₄, and any combination thereof.
20. The method of clause 1, any other suitable clause, or any combination of suitable clauses, wherein the mineral salt solution comprises KCl.
21. The method of clause 20, any other suitable clause, or any combination of suitable clauses, wherein the KCl is present at 4 mM.
22. The method of clause 20, any other suitable clause, or any combination of suitable clauses, wherein the KCl is present at 8 mM.
23. The method of clause 1, any other suitable clause, or any combination of suitable clauses, wherein the mineral salt solution comprises Na₃PO₄.
24. The method of clause 23, any other suitable clause, or any combination of suitable clauses, wherein the Na₃PO₄ is present at 20 mM.
25. The method of clause 23, any other suitable clause, or any combination of suitable clauses, wherein the Na₃PO₄ is present at 40 mM.
26. The method of clause 1, any other suitable clause, or any combination of suitable clauses, wherein the mineral salt solution comprises NH₄Cl.
27. The method of clause 26, any other suitable clause, or any combination of suitable clauses, wherein the NH₄Cl is present at 25 mM.
28. The method of clause 1, any other suitable clause, or any combination of suitable clauses, wherein the mineral salt solution comprises Na₂SO_{4.}
29. The method of clause 28, any other suitable clause, or any combination of suitable clauses, wherein the Na₂SO₄ is present at 50 mM.
30. The method of clause 1, any other suitable clause, or any combination of suitable clauses, wherein the mineral salt solution comprises KKPP, wherein KKPP comprises KCl and Na₃PO₄.
31. The method of clause 30, any other suitable clause, or any combination of suitable clauses, wherein the KCl is provided at 8 mM and the Na₃PO₄ is provided at 40 mM.
32. The method of clause 30, any other suitable clause, or any combination of suitable clauses, wherein the mineral salt solution has a stoichiometric ratio of 8 mM KCl:40 mM Na₃PO₄.
33. The method of clause 1, any other suitable clause, or any combination of suitable clauses, wherein the mineral salt solution comprises NPK, wherein NPK comprises NH₄Cl, Na₃PO₄, and KCl.
34. The method of clause 33, any other suitable clause, or any combination of suitable clauses, wherein the NH₄Cl is provided at 25 mM, the Na₃PO₄ is provided at 20 mM, and the KCl is provided at 4 mM.
35. The method of clause 33, any other suitable clause, or any combination of suitable clauses, wherein the mineral salt solution has a stoichiometric ratio of 25 mM NH₄Cl:20 mM Na₃PO₄:4 mM KCl.
36. The method of clause 1, any other suitable clause, or any combination of suitable clauses, wherein the mineral salt solution comprises KS, wherein KS comprises KCl and Na₂SO₄.
37. The method of clause 36, any other suitable clause, or any combination of suitable clauses, wherein the KCl is provided at 4 mM and the Na₂SO₄ is provided at 50 mM.
38. The method of clause 36, any other suitable clause, or any combination of suitable clauses, wherein the mineral salt solution has a stoichiometric ratio of 4 mM KCL:50 mM Na₂SO₄.
39. The method of clause 1, any other suitable clause, or any combination of suitable clauses, wherein the treated Phyla dulcis plant grows on soil.
40. The method of clause 39, any other suitable clause, or any combination of suitable clauses, wherein the soil is healthy soil.
41. The method of clause 39, any other suitable clause, or any combination of suitable clauses, wherein the soil is healthy soil comprising a combination of i) growing mix and ii) topsoil comprising organic matter.
42. The method of clause 41, any other suitable clause, or any combination of suitable clauses, wherein the growing mix and the topsoil are present at a ratio of 3:2 (v/v).
43. The method of clause 1, any other suitable clause, or any combination of suitable clauses, wherein the Phyla dulcis plant is present in a greenhouse.
44. The method of clause 1, any other suitable clause, or any combination of suitable clauses, wherein the Phyla dulcis plant is present in a bed.
45. The method of clause 1, any other suitable clause, or any combination of suitable clauses, wherein the Phyla dulcis plant is present in a field plot bed.
46. The method of clause 1, any other suitable clause, or any combination of suitable clauses, wherein the Phyla dulcis plant is present in a raised bed.
47. The method of clause 1, any other suitable clause, or any combination of suitable clauses, wherein the Phyla dulcis plant is present in a garden.
48. The method of clause 1, any other suitable clause, or any combination of suitable clauses, wherein the Phyla dulcis plant is present in a raised bed in a garden.
49. A substantially camphor-free Phyla dulcis plant produced according to the method of clauses 1 to 48.
50. The substantially camphor-free Phyla dulcis plant of clause 49, any other suitable clause, or any combination of suitable clauses, wherein the substantially camphor-free Phyla dulcis plant comprises an increased level of hernandulcin.
51. A substantially camphor-free Phyla dulcis plant part produced according to the method of clauses 1 to 48.
52. The substantially camphor-free Phyla dulcis plant part of clause 51, any other suitable clause, or any combination of suitable clauses, wherein the substantially camphor-free Phyla dulcis plant part comprises an increased level of hernandulcin.
53. The substantially camphor-free Phyla dulcis plant part of clause 51, any other suitable clause, or any combination of suitable clauses, wherein the plant part is selected from the group consisting of a seed, a flower, a leaf, and a stem.
54. The substantially camphor-free Phyla dulcis plant part of clause 51, any other suitable clause, or any combination of suitable clauses, wherein the plant part is a seed.
55. The substantially camphor-free Phyla dulcis plant part of clause 51, any other suitable clause, or any combination of suitable clauses, wherein the plant part is a flower.
56. The substantially camphor-free Phyla dulcis plant part of clause 51, any other suitable clause, or any combination of suitable clauses, wherein the plant part is a leaf.
57. The substantially camphor-free Phyla dulcis plant part of clause 51, any other suitable clause, or any combination of suitable clauses, wherein the plant part is a stem.
58. A method of producing a final Phyla dulcis plant or plant part comprising an increased level of hernandulcin, said method comprising the step of treating a Phyla dulcis plant or plant part with a mineral salt solution, wherein the treated Phyla dulcis plant or plant part grows to form the final Phyla dulcis plant or plant part comprising an increased level of hernandulcin.
59. The method of clause 58, any other suitable clause, or any combination of suitable clauses, wherein the final Phyla dulcis plant or plant part is substantially free of camphor.
60. The method of clause 58, any other suitable clause, or any combination of suitable clauses, wherein the final Phyla dulcis plant or plant part comprises an increased level of hernandulcin.
61. The method of clause 60, any other suitable clause, or any combination of suitable clauses, wherein the increased level of hernandulcin is in comparison to a comparative Phyla dulcis plant or plant part comprising camphor.
62. The method of clause 60, any other suitable clause, or any combination of suitable clauses, wherein the increased level of hernandulcin is in comparison to a comparative Phyla dulcis plant or plant part not treated with a mineral salt solution.
63. The method of clause 60, any other suitable clause, or any combination of suitable clauses, wherein the increased level of hernandulcin is greater than 7% w/w.
64. The method of clause 60, any other suitable clause, or any combination of suitable clauses, wherein the increased level of hernandulcin is greater than 6.5% w/w.
65. The method of clause 60, any other suitable clause, or any combination of suitable clauses, wherein the increased level of hernandulcin is greater than 6% w/w.
66. The method of clause 60, any other suitable clause, or any combination of suitable clauses, wherein the increased level of hernandulcin is greater than 5.5% w/w.
67. The method of clause 60, any other suitable clause, or any combination of suitable clauses, wherein the increased level of hernandulcin is greater than 5.3% w/w.
68. The method of clause 60, any other suitable clause, or any combination of suitable clauses, wherein the increased level of hernandulcin is greater than 5% w/w.
69. The method of clause 60, any other suitable clause, or any combination of suitable clauses, wherein the increased level of hernandulcin is greater than 4.5% w/w.
70. The method of clause 60, any other suitable clause, or any combination of suitable clauses, wherein the increased level of hernandulcin is greater than 4% w/w.
71. The method of clause 58, any other suitable clause, or any combination of suitable clauses, wherein the substantially camphor-free Phyla dulcis plant comprises an increased biomass in comparison to a comparative Phyla dulcis plant comprising camphor.
72. The method of clause 58, any other suitable clause, or any combination of suitable clauses, wherein the substantially camphor-free Phyla dulcis plant comprises an increased biomass in comparison to a comparative Phyla dulcis plant not treated with a mineral salt solution.
73. The method of clause 58, any other suitable clause, or any combination of suitable clauses, wherein the final Phyla dulcis plant or plant part comprises an antioxidant.
74. The method of clause 73, any other suitable clause, or any combination of suitable clauses, wherein the antioxidant is a shikimal-based antioxidant.
75. The method of clause 58, any other suitable clause, or any combination of suitable clauses, wherein the mineral salt solution comprises an element selected from the group consisting of K, Cl, Na, P, O, N, H, S, and any combination thereof.
76. The method of clause 58, any other suitable clause, or any combination of suitable clauses, wherein the mineral salt solution comprises an element selected from the group consisting of K, P, N, S, and any combination thereof.
77. The method of clause 58, any other suitable clause, or any combination of suitable clauses, wherein the mineral salt solution comprises a formulation selected from the group consisting of KCl, Na₃PO₄, NH₄Cl, Na₂SO₄, and any combination thereof.
78. The method of clause 58, any other suitable clause, or any combination of suitable clauses, wherein the mineral salt solution comprises KCl.
79. The method of clause 78, any other suitable clause, or any combination of suitable clauses, wherein the KCl is present at 4 mM.
80. The method of clause 78, any other suitable clause, or any combination of suitable clauses, wherein the KCl is present at 8 mM.
81. The method of clause 58, any other suitable clause, or any combination of suitable clauses, wherein the mineral salt solution comprises Na₃PO₄.
82. The method of clause 81, any other suitable clause, or any combination of suitable clauses, wherein the Na₃PO₄ is present at 20 mM.
83. The method of clause 81, any other suitable clause, or any combination of suitable clauses, wherein the Na₃PO₄ is present at 40 mM.
84. The method of clause 58, any other suitable clause, or any combination of suitable clauses, wherein the mineral salt solution comprises NH₄Cl.
85. The method of clause 84, any other suitable clause, or any combination of suitable clauses, wherein the NH₄Cl is present at 25 mM.
86. The method of clause 58, any other suitable clause, or any combination of suitable clauses, wherein the mineral salt solution comprises Na₂SO₄.
87. The method of clause 86, any other suitable clause, or any combination of suitable clauses, wherein the Na₂SO₄ is present at 50 mM.
88. The method of clause 58, any other suitable clause, or any combination of suitable clauses, wherein the mineral salt solution comprises KKPP, wherein KKPP comprises KCl and Na₃PO₄.
89. The method of clause 88, any other suitable clause, or any combination of suitable clauses, wherein the KCl is provided at 8 mM and the Na₃PO₄ is provided at 40 mM.
90. The method of clause 88, any other suitable clause, or any combination of suitable clauses, wherein the mineral salt solution has a stoichiometric ratio of 8 mM KCl:40 mM Na₃PO₄.
91. The method of clause 58, any other suitable clause, or any combination of suitable clauses, wherein the mineral salt solution comprises NPK, wherein NPK comprises NH₄Cl, Na₃PO₄, and KCl.
92. The method of clause 91, any other suitable clause, or any combination of suitable clauses, wherein the NH₄Cl is provided at 25 mM, the Na₃PO₄ is provided at 20 mM, and the KCl is provided at 4 mM.
93. The method of clause 91, any other suitable clause, or any combination of suitable clauses, wherein the mineral salt solution has a stoichiometric ratio of 25 mM NH₄Cl:20 mM Na₃PO₄:4 mM KCl.
94. The method of clause 58, any other suitable clause, or any combination of suitable clauses, wherein the mineral salt solution comprises KS, wherein KS comprises KCl and Na₂SO₄.
95. The method of clause 94, any other suitable clause, or any combination of suitable clauses, wherein the KCl is provided at 4 mM and the Na₂SO₄ is provided at 50 mM.
96. The method of clause 94, any other suitable clause, or any combination of suitable clauses, wherein the mineral salt solution has a stoichiometric ratio of 4 mM KCL:50 mM Na₂SO₄.
97. The method of clause 58, any other suitable clause, or any combination of suitable clauses, wherein the treated Phyla dulcis plant grows on soil.
98. The method of clause 97, any other suitable clause, or any combination of suitable clauses, wherein the soil is healthy soil.
99. The method of clause 97, any other suitable clause, or any combination of suitable clauses, wherein the soil is healthy soil comprising a combination of i) growing mix and ii) topsoil comprising organic matter.
100. The method of clause 99, any other suitable clause, or any combination of suitable clauses, wherein the growing mix and the topsoil are present at a ratio of 3:2 (v/v).
101. The method of clause 58, any other suitable clause, or any combination of suitable clauses, wherein the Phyla dulcis plant is present in a greenhouse.
102. The method of clause 58, any other suitable clause, or any combination of suitable clauses, wherein the Phyla dulcis plant is present in a bed.
103. The method of clause 58, any other suitable clause, or any combination of suitable clauses, wherein the Phyla dulcis plant is present in a field plot bed.
104. The method of clause 58, any other suitable clause, or any combination of suitable clauses, wherein the Phyla dulcis plant is present in a raised bed.
105. The method of clause 58, any other suitable clause, or any combination of suitable clauses, wherein the Phyla dulcis plant is present in a garden.
106. The method of clause 58, any other suitable clause, or any combination of suitable clauses, wherein the Phyla dulcis plant is present in a raised bed in a garden.
107. A Phyla dulcis plant produced according to the method of any one of clauses 58 to 106.
108. The Phyla dulcis plant of clause 107, any other suitable clause, or any combination of suitable clauses, wherein the Phyla dulcis plant comprises an increased level of hernandulcin.
109. A Phyla dulcis plant part produced according to the method of clauses 58 to 106.
110. The Phyla dulcis plant part of clause 109, any other suitable clause, or any combination of suitable clauses, wherein the Phyla dulcis plant part comprises an increased level of hernandulcin.
111. The Phyla dulcis plant part of clause 109, any other suitable clause, or any combination of suitable clauses, wherein the plant part is selected from the group consisting of a seed, a flower, a leaf, and a stem.
112. The Phyla dulcis plant part of clause 109, any other suitable clause, or any combination of suitable clauses, wherein the plant part is a seed.
113. The Phyla dulcis plant part of clause 109, any other suitable clause, or any combination of suitable clauses, wherein the plant part is a flower.
114. The Phyla dulcis plant part of clause 109, any other suitable clause, or any combination of suitable clauses, wherein the plant part is a leaf.
115. The Phyla dulcis plant part of clause 109, any other suitable clause, or any combination of suitable clauses, wherein the plant part is a stem.
116. A composition comprising hernandulcin, wherein the hernandulcin is produced according to the method of any of one of clauses 1 to 48 or any one of clauses 58 to 106.
117. The composition of clause 116, any other suitable clause, or any combination of suitable clauses, wherein the composition is configured for inclusion in a beverage.
118. The composition of clause 117, any other suitable clause, or any combination of suitable clauses, wherein the beverage is selected from the group consisting of a soft drink beverage, a bottled beverage, an alcoholic beverage, a non-alcoholic beverage, a weight-loss beverage, and water.
119. The composition of clause 116, any other suitable clause, or any combination of suitable clauses, wherein the composition is configured for inclusion in a confectionary.
120. The composition of clause 119, any other suitable clause, or any combination of suitable clauses, wherein the confectionary is selected from the group consisting of a jelly, a syrup, a honey, a candy, a chewing gum, and any combination thereof.
121. The composition of clause 116, any other suitable clause, or any combination of suitable clauses, wherein the composition is configured for inclusion in a food.
122. The composition of clause 121, any other suitable clause, or any combination of suitable clauses, wherein the food is selected from the group consisting of a bread, a cake, a cookie, a salad dressing, a corn flake, a rice flake, and any combination thereof.
123. The composition of clause 116, any other suitable clause, or any combination of suitable clauses, wherein the composition is a dietary supplement.
124. The composition of clause 116, any other suitable clause, or any combination of suitable clauses, wherein the composition is configured for inclusion in a formulation selected from the group consisting of a sugar-free malt drink, a sugar-free soft drink, a sugar-free beer, a sugar-free ice cream, a sugar-free yogurt, a sugar-free toothpaste, a sugar-free iced tea, a sugar-free jelly, a sugar-free candy, a sugar-free bottled water, a sugar-free fruit juice, a sugar-free marmalade, a sugar-free chewing gum, a sugar-free mouth wash, and a sugar-free tomato ketchup.
125. The composition of clause 116, any other suitable clause, or any combination of suitable clauses, wherein the composition is configured for inclusion in a sugar-free malt drink.
126. The composition of clause 116, any other suitable clause, or any combination of suitable clauses, wherein the composition is configured for inclusion in a sugar-free soft drink.
127. The composition of clause 116, any other suitable clause, or any combination of suitable clauses, wherein the composition is configured for inclusion in a sugar-free beer.
128. The composition of clause 116, any other suitable clause, or any combination of suitable clauses, wherein the composition is configured for inclusion in a sugar-free ice cream.
129. The composition of clause 116, any other suitable clause, or any combination of suitable clauses, wherein the composition is configured for inclusion in a sugar-free yogurt.
130. The composition of clause 116, any other suitable clause, or any combination of suitable clauses, wherein the composition is configured for inclusion in a sugar-free toothpaste.
131. The composition of clause 116, any other suitable clause, or any combination of suitable clauses, wherein the composition is configured for inclusion in a sugar-free iced tea.
132. The composition of clause 116, any other suitable clause, or any combination of suitable clauses, wherein the composition is configured for inclusion in a sugar-free jelly.
133. The composition of clause 116, any other suitable clause, or any combination of suitable clauses, wherein the composition is configured for inclusion in a sugar-free candy.
134. The composition of clause 116, any other suitable clause, or any combination of suitable clauses, wherein the composition is configured for inclusion in a sugar-free bottled water.
135. The composition of clause 116, any other suitable clause, or any combination of suitable clauses, wherein the composition is configured for inclusion in a sugar-free fruit juice.
136. The composition of clause 116, any other suitable clause, or any combination of suitable clauses, wherein the composition is configured for inclusion in a sugar-free marmalade.
137. The composition of clause 116, any other suitable clause, or any combination of suitable clauses, wherein the composition is configured for inclusion in a sugar-free chewing gum.
138. The composition of clause 116, any other suitable clause, or any combination of suitable clauses, wherein the composition is configured for inclusion in a sugar-free mouth wash.
139. The composition of clause 116, any other suitable clause, or any combination of suitable clauses, wherein the composition is configured for inclusion in a sugar-free tomato ketchup.

### EXAMPLE

### Exemplary Method of Producing Substantially Camphor-free Phyla dulcis Plant

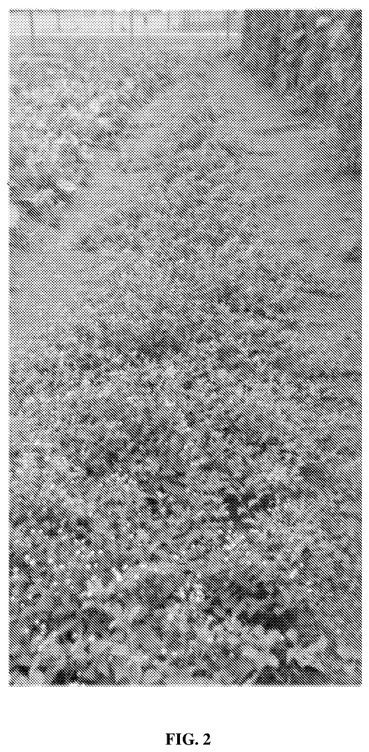

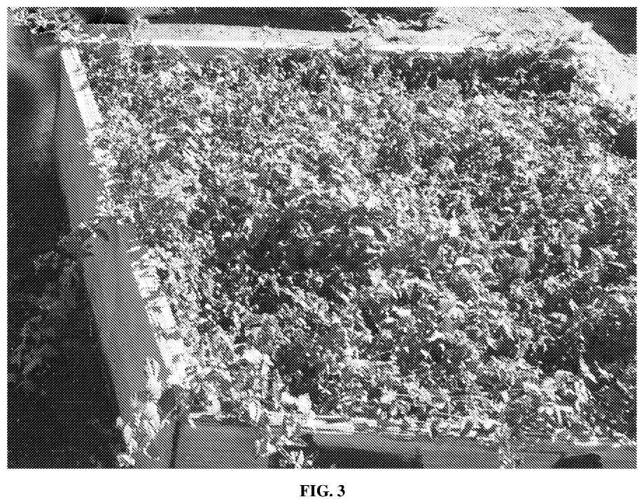

In the instant example, an exemplary method for producing the Phyla dulcis plant of the present disclosure is provided. P. dulcis was grown in Texas, USA as it can grow very well in the spring and early summer (see FIG. 2). The plant may need some shading in order to continue growing through a hot summer until early fall. For instance, the plant can grow to a high density per unit area of land as shown in FIG. 3 in which the plants over-grew their raised beds.

For the instant example, P. dulcis stem cuttings were planted in raised beds (approximately 120×120×30 cm: width×length×depth) in April and treated with a mineral salt solution of various stoichiometric combinations of salts of Nitrogen (N), Potassium (K), Sulfur (S), and Phosphorus (P). A control plant group was treated with water. The mineral salt solution was applied 2 or 3 times per cropping season at about 2 liters per bed.

The beds contained identical healthy soil: 2 bags of top soil mixed with 3 bags of growing mix, Plants watered equally every other day. No insect pests were observed. Weather conditions (temperature, humidity, rainfall, solar irradiance, wind speed) were recorded.

P. dulcis begins to flower about 3-4 weeks after planting and attracts many pollinators. Therefore, thousands of seeds are produced with the P. dulcis plant and the seeds contain hernandulcin.

Generally, the seeds, flowers (inflorescence), leaves, and stems of P. dulcis plants can contain hernandulcin and camphor. Upon maturity in November, the P. dulcis shoots, flowers, and seeds were harvested, weighed, and immediately stored at 5° C. in a walk-in refrigerator in order to allow terpinoid biochemical reactions to exhaust all intermediates. Temporary storage of the biomass in the fridge did not decrease the hernandulcin concentration. The entire harvest for each bed was ground to coarse powder, freeze-dried, and stored at −80° C. Plant samples were then extracted for analyses via high pressure liquid chromatographic (HPLC) and GC-MS in order to quantitate concentrations of hernandulcin and camphor,

Results from the instant example are found in Table 1, which shows HPLC analyses of the hernandulcin and camphor concentrations of various plants. As shown in Table 1, four groups were tested: a P. dulcis plant control group and P. dulcis plants that were administered one of three different stoichiometric mixes of mineral salt solutions. As shown in Table 1, leaves from P. dulcis control plants contain higher concentrations of camphor, lower concentrations of hernandulcin, and shikimic acid together with other antioxidants (see Osuji et al., 2017).

TABLE 1

| Mineral Salt | Biomass per ha | Camphor % | Hernandulcin % |
|---|---|---|---|
| Solution | (kg) | (w/w) | (w/w) |
| Control | 5921 | 4.28 | 5.30 |
| KKPP* | 1884 | <0.001 | 13.56 |
| NPK** | 6862 | <0.002 | 13.64 |
| KS*** | 23,546 | <0.001 | 16.08 |

*KCl (8 mM) and Na₃PO₄ (40 mM)
**NH₄Cl (25 mM), Na₃PO₄ (20 mM), and KCl (4 mM)
***KCl (4 mM) and Na₂SO₄ (50 mM)

Table 1 demonstrates that the application of mineral salt solutions affected three different properties of the molecular chemistry of Phyla dulcis plants: biomass growth, monoterpene biosynthesis, and sesquiterpene biosynthesis. For instance, biomass growth was shown to increase up to 400% compared to control with the application of the KS mineral salt solution.

Importantly, the application of mineral salt solutions to the Phyla dulcis plants demonstrated a significant reduction in camphor and a large increase in hernandulcin. With the suppression of camphor synthesis, hernandulcin synthesis became optimized in the stoichiometric mineral salts-treated P. dulcis plants (e.g., up to a 300% increase with application of the KS mineral salt solution). Concentrations of hernandulcin as shown in Table 1 are above the control of 5.3% w/w. Furthermore, the concentrations of hernandulcin as shown in Table 1 are dramatically higher than the larges yield of hernandulcin reported from natural growing P. dulcis plants (0.154%; see Kinghorn and Soejarto, 2002).

In summary, the instant example demonstrates that application of mineral salt solutions to Phyla dulcis plants reduced camphor (monoterpene) accumulation and enhanced the hernandulcin (sesquiterpene) accumulation in the plants and plant parts. Furthermore, as shown in FIGS. 2 and 3, scale-up of the cultivation to industrial levels is horticulturally sustainable and mineral salt solutions can easily be applied to such plants.

1. A method of producing a substantially camphor-free Phyla dulcis plant, said method comprising the step of treating a Phyla dulcis plant with a mineral salt solution, wherein the treated Phyla dulcis plant grows to form the substantially camphor-free Phyla dulcis plant.

2. The method of claim 1, wherein the substantially camphor-free Phyla dulcis plant comprises an increased level of hernandulcin.

3. The method of claim 2, wherein the increased level of hernandulcin is in comparison to a comparative Phyla dulcis plant comprising camphor.

4. The method of claim 2, wherein the increased level of hernandulcin is in comparison to a comparative Phyla dulcis plant not treated with a mineral salt solution.

5. The method of claim 2, wherein the increased level of hernandulcin is greater than 5.3% w/w.

6. The method of claim 2, wherein the increased level of hernandulcin is greater than 4% w/w.

7. The method of claim 1, wherein the substantially camphor-free Phyla dulcis plant comprises an increased biomass in comparison to a comparative Phyla dulcis plant comprising camphor.

8. The method of claim 1, wherein the substantially camphor-free Phyla dulcis plant comprises an increased biomass in comparison to a comparative Phyla dulcis plant not treated with a mineral salt solution.

9. The method of claim 1, wherein the mineral salt solution comprises an element selected from the group consisting of K, P, N, S, and any combination thereof.

10. The method of claim 1, wherein the mineral salt solution comprises a formulation selected from the group consisting of KCl, Na₃PO₄, NH₄Cl, Na₂SO₄, and any combination thereof.

11. The method of claim 1, wherein the mineral salt solution comprises KKPP, wherein KKPP comprises KCl and Na₃PO₄.

12. The method of claim 11, wherein the KCl is provided at 8 mM and the Na₃PO₄ is provided at 40 mM.

13. The method of claim 11, wherein the mineral salt solution has a stoichiometric ratio of 8 mM KCl:40 mM Na₃PO₄.

14. The method of claim 1, wherein the mineral salt solution comprises NPK, wherein NPK comprises NH₄Cl, Na₃PO₄, and KCl.

15. The method of claim 14, wherein the NH₄Cl is provided at 25 mM, the Na₃PO₄ is provided at 20 mM, and the KCl is provided at 4 mM.

16. The method of claim 14, wherein the mineral salt solution has a stoichiometric ratio of 25 mM NH₄Cl:20 mM Na₃PO₄:4 mM KCl.

17. The method of claim 1, wherein the mineral salt solution comprises KS, wherein KS comprises KCl and Na₂SO₄.

18. The method of claim 17, wherein the KCl is provided at 4 mM and the Na₂SO₄ is provided at 50 mM.

19. The method of claim 17, wherein the mineral salt solution has a stoichiometric ratio of 4 mM KCL:50 mM Na₂SO₄.

20. A substantially camphor-free Phyla dulcis plant produced according to the method of claim 1.

21-57. (canceled)
